(19) United States
(12) Patent Application Publication
(10) Pub. No.: US 2022/0409008 A1
(43) Pub. Date: Dec. 29, 2022

(54) ENDOSCOPE

(71) Applicant: OLYMPUS CORPORATION, Tokyo (JP)

(72) Inventor: Takahiro SUWA, Tokyo (JP)

(73) Assignee: OLYMPUS CORPORATION, Tokyo (JP)

(21) Appl. No.: 17/901,202

(22) Filed: Sep. 1, 2022

Related U.S. Application Data
(63) Continuation of application No. PCT/JP2020/011278, filed on Mar. 13, 2020.

(51) Int. Cl. A61B 1/005; A61B 1/00

(52) U.S. Cl. CPC A61B 1/005; A61B 1/00071; A61B 1/0011

(57) ABSTRACT

An endoscope includes: an operating portion; an insertion tube that has a central axis; a bendable tube that is bendable in a curvature direction orthogonal to the central axis, the bendable tube including a plurality of bendable tube pieces that are arrayed along the central axis, adjacent bendable tube pieces being connected to each other by a pair of connection portions, the pair of connection portions being arranged line-symmetric with respect to the curvature direction; a distal end rigid portion that is provided on a distal end side of the bendable tube; and a wire having a first end connected to the operating portion, and a second end connected to either the bendable tube or the distal end rigid portion.

### CROSS-REFERENCE TO RELATED APPLICATION

This application is a continuation of International Application No. PCT/JP2020/011278, filed on Mar. 13, 2020, the entire contents of which are incorporated herein by reference.

### BACKGROUND

### 1. Technical Field

The present disclosure relates to an endoscope.

### 2. Related Art

In the related art, an endoscope has performed the acquisition of image data of the inside of a subject using an imager, and treatment using a processing tool or the like, by inserting an insertion portion into a subject such as a patient. A bendable tube of the insertion portion of the endoscope includes a plurality of bendable tube pieces, and is made bendable by operating a wire inserted into the inside of the bendable tube pieces. Then, the endoscope enables a viewing field to be changed, by bending the bendable tube.

Because the insertion portion of the endoscope is inserted into a narrow digestive tract or the like, an accidental symptom such as perforation might occur by bending the bendable tube. For preventing the body of a patient from being damaged in this manner, an endoscope for a digestive tract preferably has a small bending height (for example, refer to a bending height H in FIG. 6E.

JP 2005-237609 A discusses an endoscope in which an additional tube is bonded to the proximal end side of a bendable tube using adhesive in such a manner as to make the proximal end side hard to be bent, and cause bending from the distal end side. By causing the bendable tube to bend from the distal end side, the bending height H can be made smaller. In this manner, by employing a bending shape suitable for the intended purpose of an endoscope, the operability of the endoscope can be enhanced.

### SUMMARY

In some embodiments, an endoscope includes: an operating portion configured to receive a bending operation performed by an operator; an insertion tube that is provided on the operating portion and that has a central axis; a bendable tube that is provided on a distal end side of the insertion tube and that is bendable in a curvature direction orthogonal to the central axis, the bendable tube including a plurality of bendable tube pieces that are arrayed along the central axis, adjacent bendable tube pieces being connected to each other by a pair of connection portions, the pair of connection portions being arranged line-symmetric with respect to the curvature direction, the bendable tube being configured to allow different positions in the curvature direction of the adjacent bendable tube pieces that are at least part of the plurality of bendable tube pieces; a distal end rigid portion that is provided on a distal end side of the bendable tube; and a wire having a first end connected to the operating portion, and a second end connected to either the bendable tube or the distal end rigid portion.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram schematically illustrating an endoscope system according to an embodiment;

FIG. 2 is a perspective view schematically illustrating a configuration of a distal end of an insertion portion of an ultrasound endoscope according to an embodiment;

FIG. 3 is a side view of the distal end of the insertion portion;

FIG. 4 is a side view of bendable tube pieces;

FIG. 5 is a cross-sectional view corresponding to an A-A line in FIG. 4;

FIGS. 6A to 6E are diagrams illustrating a state in which a bendable tube bends;

FIG. 7 is a perspective view of a bendable tube according to a modified example; and

FIG. 8 is a diagram illustrating positions of connection portions and a wire in a bendable tube.

### DETAILED DESCRIPTION

Hereinafter, embodiments of an endoscope according to the disclosure will be described with reference to the drawings. In addition, the disclosure is not limited by these embodiments. The disclosure can be generally applied to an endoscope having a bendable distal end.

In addition, in the description of the drawings, the same or corresponding elements are appropriately assigned the same signs. In addition, it should also be noted that the drawings are schematic drawings, and a dimensional relationship between elements, proportion of elements, and the like sometimes differ from reality. Also in the drawings, parts mutually-different in dimensional relationship and proportion are sometimes included.

### EMBODIMENT

Configuration of Endoscope System

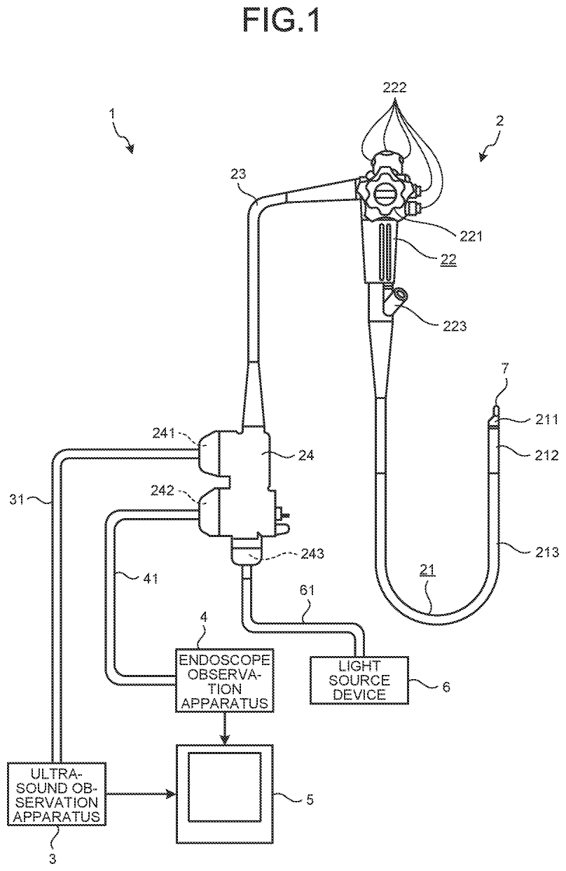

FIG. 1 is a diagram schematically illustrating an endoscope system according to an embodiment. An endoscope system 1 is a system for performing ultrasound diagnosis of the inside of a subject such as a human, using an ultrasound endoscope. As illustrated in FIG. 1, the endoscope system 1 includes an ultrasound endoscope 2, an ultrasound observation apparatus 3, an endoscope observation apparatus 4, a display device 5, and a light source device 6.

Using an ultrasound transducer provided at its distal end portion, the ultrasound endoscope 2 converts an electrical pulse signal received from the ultrasound observation apparatus 3, into an ultrasound pulse, emits the ultrasound pulse onto a subject, converts an ultrasound echo reflected by the subject, into an electrical echo signal represented by voltage change, and outputs the electrical echo signal.

The ultrasound endoscope 2 normally includes an imaging optical system and an image sensor, is inserted into a digestive tract (esophagus, stomach, duodenum, large intestine) or respiratory organs (trachea, bronchus) of the subject, and can perform imaging of the digestive tract or any of the respiratory organs. In addition, their adjacent organs (pancreas, cholecystis, biliary duct, biliary tract, lymph nodes, mediastinum organ, blood vessels, etc.) can be captured using ultrasound waves. In addition, the ultrasound endoscope 2 includes a light guide that guides illumination light to be emitted onto a subject when optical imaging is performed. This light guide has a distal end portion and a proximal end portion. While the distal end portion reaches a distal end of an insertion portion to be inserted into a subject of the ultrasound endoscope 2, the proximal end portion is connected to the light source device 6 that generates illumination light.

As illustrated in FIG. 1, the ultrasound endoscope 2 includes an insertion portion 21, an operating portion 22, a universal cord 23, and a connector 24. The insertion portion 21 is a portion to be inserted into the subject. As illustrated in FIG. 1, this insertion portion 21 is provided on the distal end side, and includes a distal end rigid portion 211 holding a convex-type ultrasound transducer 7, a bendable tube 212 that is bendable in a curvature direction orthogonal to a central axis, and a flexible insertion tube 213 having a central axis. The distal end rigid portion 211 is provided on the bendable tube 212, the bendable tube 212 is provided on the distal end side of the insertion tube 213, and the insertion tube 213 is provided on the distal end side of the operating portion 22. Here, on the inside the insertion portion 21, a light guide for transferring illumination light supplied from the light source device 6, and a plurality of signal cables for transferring various signals are laid, and a processing tool insertion path or the like for inserting a processing tool is formed, of which specific illustrations are omitted in the drawings. The configuration of the distal end of the insertion portion 21 and the configuration of the bendable tube 212 will be described later.

The operating portion 22 is a portion that is connected to a proximal end side of the insertion portion 21, and receives various operations from an operator such as a doctor. As illustrated in FIG. 1, this operating portion 22 includes a bending knob 221 for receiving a bending operation of bending the bendable tube 212, and a plurality of operating members 222 for performing various operations. In addition, a processing tool insertion port 223 that is communicated with the processing tool insertion path, and is provided for inserting a processing tool into the processing tool insertion path is formed on the operating portion 22.

The universal cord 23 is a cable extending from the operating portion 22, and in which a plurality of signal cables for transferring various signals, optical fibers for transferring illumination light supplied from the light source device 6, and the like are laid.

The connector 24 is provided at a distal end of the universal cord 23. Then, the connector 24 includes a first connector portion 241 to which an ultrasound cable 31 is connected, a second connector portion 242 to which a video cable 41 is connected, and a third connector portion 243 to which an optical fiber cable 61 is connected.

The ultrasound observation apparatus 3 electrically-connects to the ultrasound endoscope 2 via the ultrasound cable 31 (refer to FIG. 1), outputs a pulse signal to the ultrasound endoscope 2 via the ultrasound cable 31, and receives an echo signal input from the ultrasound endoscope 2. Then, the ultrasound observation apparatus 3 performs predetermined processing on the echo signal to generate an ultrasound image.

The endoscope observation apparatus 4 electrically-connects to the ultrasound endoscope 2 via the video cable 41 (refer to FIG. 1), and inputs an image signal from the ultrasound endoscope 2 via the video cable 41. Then, the endoscope observation apparatus 4 performs predetermined processing on the image signal to generate an endoscope image.

The display device 5 is formed by using a liquid crystal, an organic electro luminescence (EL), a projector, a cathode ray tube (CRT), or the like, and displays the ultrasound image generated in the ultrasound observation apparatus 3, the endoscope image generated in the endoscope observation apparatus 4, and the like.

The light source device 6 connects to the ultrasound endoscope 2 via the optical fiber cable 61 (refer to FIG. 1), and supplies, via the optical fiber cable 61, illumination light for illuminating the inside of a subject, to the ultrasound endoscope 2.

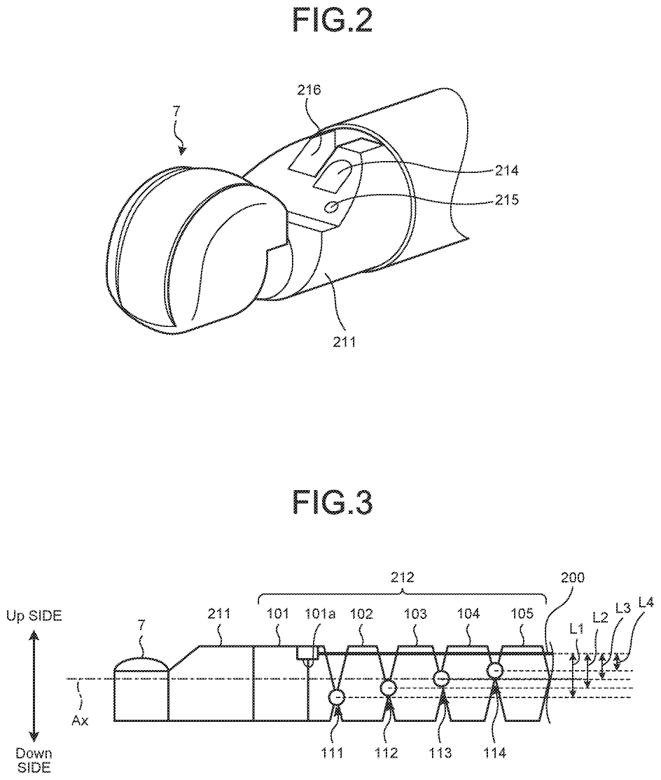

Configuration of Distal End of Insertion Portion FIG. 2 is a perspective view schematically illustrating a configuration of a distal end of an insertion portion of an ultrasound endoscope according to an embodiment. As illustrated in FIG. 2, the ultrasound endoscope 2 includes an imager 214 that generates an image by imaging a subject being an observation object, from an objective lens provided on the distal end rigid portion 211, an illumination unit 215 that emits illumination light into the subject from an illumination lens provided on the distal end rigid portion 211, and a processing tool protrusion port 216 that is communicated with the processing tool insertion path formed in the insertion portion 21, and protrudes a processing tool from the distal end of the insertion portion 21.

Configuration of Bendable Tube

FIG. 3 is a side view of the distal end of the insertion portion. As illustrated in FIG. 3, the bendable tube 212 includes bendable tube pieces 101 to 105 arrayed along a central axis Ax of a cylinder formed by the bendable tube 212. The bendable tube 212 is only required to include a plurality of bendable tube pieces, and the number of bendable tube pieces is not specifically limited. The bendable tube 212 is bendable toward an Up side corresponding to an upper direction of an image captured by the imager 214, and a Down side being an opposite side of the Up side. In addition, the ultrasound endoscope 2 includes a wire 200 having one end fixed to the bendable tube piece 101 positioned closest to the distal end side. FIG. 3 illustrates the wire 200 for the sake of explanation, but the wire 200 is invisible from the side because the wire 200 is inserted into the bendable tube pieces 101 to 105. In addition, the wire 200 may be fixed to the distal end rigid portion 211 or a bendable tube piece other than the bendable tube piece 101.

The bendable tube pieces 101 to 105 are connected by corresponding pairs of connection portions 111 to 114 provided on the front side and rear side in FIG. 3. The positions in a curvature direction (direction extending along the Up side and the Down side) of the connection portions 111 to 114 are positions getting away from the wire 200 as getting closer to the distal end side (connection portion 111 side) of the bendable tube 212. In other words, a relationship of a distance L1 between the connection portion 111 and the wire 200>a distance L2 between the connection portion 112 and the wire 200>a distance L3 between the connection portion 113 and the wire 200>a distance L4 between the connection portion 114 and the wire 200 is satisfied.

The bendable tube piece 101 includes a wire fixing portion 101a, and an end portion on the distal end side of the wire 200 is fixed to the wire fixing portion 101a.

At an end portion on the proximal end side, the wire 200 is connected to the bending knob 221 of the operating portion 22. If the bending knob 221 is rotated, the wire 200 is pulled toward the proximal end side, and the bendable tube 212 is bent in a predetermined curvature direction (Up side).

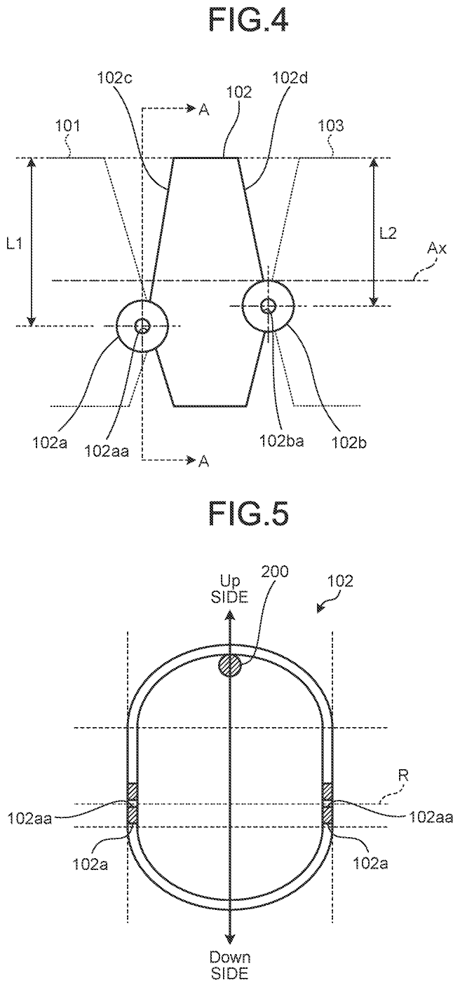

FIG. 4 is a side view of bendable tube pieces. As illustrated in FIG. 4, the bendable tube piece 102 includes a pair of connection surfaces 102a provided on the distal end side, a pair of connection surfaces 102b provided on the proximal end side, a taper surface 102c provided on the distal end side, and a taper surface 102d provided on the proximal end side.

FIG. 5 is a cross-sectional view corresponding to an A-A line in FIG. 4. As illustrated in FIG. 5, the pair of connection surfaces 102a face each other and are parallel to each other. In other words, the pair of connection surfaces 102a are arranged in such a manner that their positions in the curvature direction become the same on the pair of connection surfaces 102a, and become line-symmetric with respect to the curvature direction. The pair of connection surfaces 102a are overlaid on a pair of connection surfaces provided on the proximal end side of the bendable tube piece 101, and by pins being respectively inserted into two holes 102aa, function as the connection portion 111 being a hinge. As a result of this, the bendable tube piece 101 and the bendable tube piece 102 are connected by the pair of connection portions 111 in such a manner as to be rotatable about a rotation axis R Accordingly, the pair of connection portions 111 face each other and are parallel to each other. In other words, the positions in the curvature direction of the pair of connection portions 111 are the same in the pair of connection portions 111.

Similarly, the pair of connection surfaces 102b are overlaid on a pair of connection surfaces provided on the distal end side of the bendable tube piece 103, and by pins being respectively inserted into two holes 102ba, function as the connection portion 112 being a hinge. As a result of this, the bendable tube piece 102 and the bendable tube piece 103 are rotatably connected by the pair of connection portions 112.

Referring back to FIG. 4, the taper surface 102c is formed in such a manner that a width of the bendable tube piece 102 in a direction extending along a central axis Ax decreases along the curvature direction (Up side) from the connection surface 102a (connection portion 111). As a result of this, a gap between the bendable tube piece 102 and the bendable tube piece 101 becomes larger as proceeding in the curvature direction (Up side), and the bendable tube piece 102 and the bendable tube piece 101 are prevented from interfering with each other when the bendable tube 212 bends.

Similarly, the taper surface 102d is formed in such a manner that a width of the bendable tube piece 102 in a direction extending along the central axis Ax decreases along the curvature direction (Up side) from the connection surface 102b (connection portion 112). As a result of this, a gap between the bendable tube piece 102 and the bendable tube piece 103 becomes larger as proceeding in the curvature direction (Up side), and the bendable tube piece 102 and the bendable tube piece 103 are prevented from interfering with each other when the bendable tube 212 bends.

In addition, the bendable tube pieces 101, 103, 104, and 105 also include similar connection surfaces and taper surfaces, but have similar configurations except that positions in the curvature directions of the connection surfaces are different. Thus, the description will be omitted.

In addition, in the bendable tube piece 102, the taper surface 102c is formed on the distal end side and the taper surface 102d is formed on the proximal end side, but a taper surface may be formed on either one of the distal end side or the proximal end side.

Bending Way of Bendable Tube

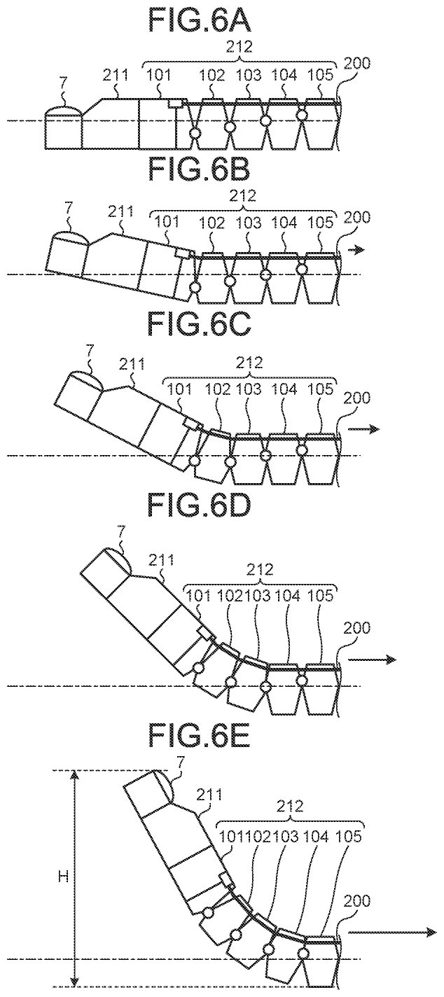

FIGS. 6A to 6E are diagram illustrating a state in which a bendable tube bends. As illustrated in FIGS. 6A to 6E, if the wire 200 is pulled toward the proximal end side, the connection portion 111, the connection portion 112, the connection portion 113, and the connection portion 114 bend in this order.

Torques of connection portions 111 to 114 are obtained by products of force of pulling the wire 200, and the distances L1 to L4 between the connection portions 111 to 114 and the wire 200. Because the distance L1>the distance L2>the distance L3>the distance L4 is satisfied, the torque of the connection portion 111 is largest and the connection portions rotate in order from the connection portion 111. As a result of this, a bending height H illustrated in FIG. 6E can be made smaller than that of a conventional endoscope.

According to the embodiment described above, by the connection portions rotating in order from the connection portion on the distal end side, the ultrasound endoscope 2 with a small bending height H can be realized. Because this ultrasound endoscope 2 does not require a tube or the like that makes the bendable tube 212 hard to bend, the operability is maintained. In addition, in a case where this ultrasound endoscope 2 is used for a digestive tract, because the ultrasound endoscope 2 has a small bending height H, it is possible to suppress the occurrence of an accidental symptom such as perforation.

In addition, in the above-described embodiment, an example in which the connection portions rotate from the connection portion on the distal end side has been described, but it is possible to realize an endoscope in which the connection portions rotate from a desired position in accordance with the intended purpose of the endoscope. For example, by setting the positions in the curvature direction of connection portions to positions getting away from the wire 200 as getting closer to the proximal end side (connection portion 114 side) of the bendable tube 212, it is possible to realize an endoscope in which the connection portions rotate from the connection portion on the proximal end side. If a radius of a bendable tube is denoted by ϕ, in the conventional endoscope, a connection portion has been provided at a position at which a distance L0 between the connection portion and a wire satisfies L0=ϕ/2. Accordingly, at a position at which a distance between a connection portion and a wire is larger than ϕ/2, the connection portion rotates with force smaller than that in the prior art, and the connection portion bends more easily. Accordingly, at a position at which a distance between a connection portion and a wire is smaller than ϕ/2, the connection portion rotates with force larger than that in the prior art, and the connection portion gets hard to bend. By appropriately selecting a distance between a connection portion and a wire, and differentiating positions in the curvature direction of adjacent bendable tube pieces that are at least part of the bendable tube pieces, it is possible to realize an endoscope that is bendable from a desired position in accordance with the intended purpose of the endoscope.

### Modified Example

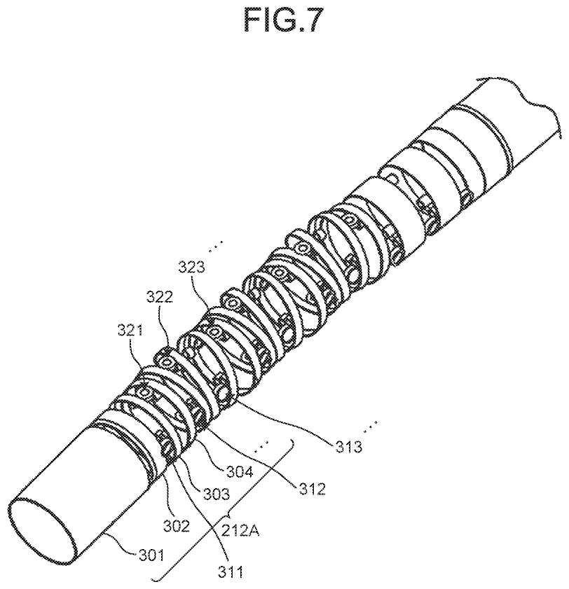

Next, a bendable tube of an endoscope according to a modified example will be described. FIG. 7 is a perspective view of a bendable tube according to a modified example. A bendable tube 212A includes a plurality of bendable tube pieces including bendable tube pieces 301 to 304, and these bendable tube pieces are connected by pairs of connection portions 311 to 313 and pairs of connection portions 321 to 323. The connection portions 311 to 313 and the connection portions 321 to 323 are ball joints, for example, and are bendable in an arbitrary direction. In this case, the pairs of connection portions 311 to 313 and the pairs of connection portions 321 to 323 need not exist at positions in the bendable tube 212A at which connection portions face each other, and need not be arranged in parallel to each other.

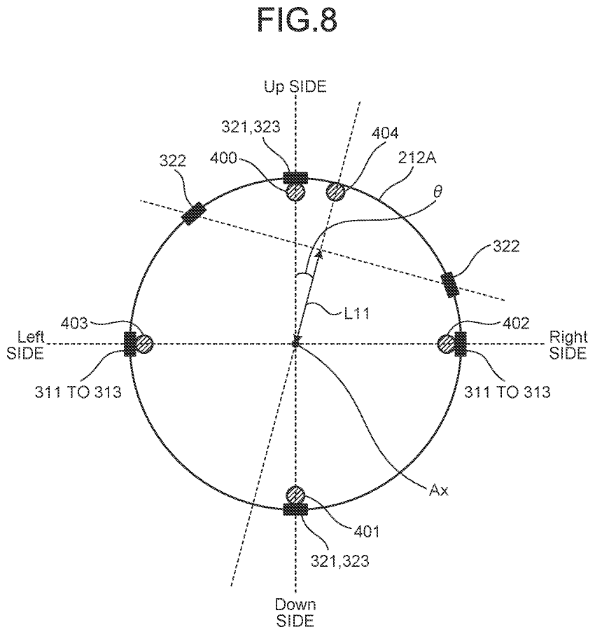

FIG. 8 is a diagram illustrating positions of connection portions and a wire in a bendable tube. FIG. 8 is a diagram illustrating the bendable tube 212A viewed from the distal end side. The pair of connection portions 311 to 313 are arranged to face each other with centering around a central axis Ax, in a direction extending along a Right side and a Left side. The pair of connection portions 321 and 323 are arranged to face each other with centering around the central axis Ax, in a direction extending along an Up side and a Down side. As a result of this, the bendable tube 212A is bendable in four directions corresponding to the Up side, the Down side, the Right side, and the Left side.

A pair of connection portions 322 are arranged at symmetric positions about a point shifted from the central axis Ax by a distance L11, around a direction shifted by predetermined angle θ from the direction extending along the Up side and the Down side. In this manner, by arranging the pair of connection portions 322 with being shifted toward a wire 404 side, it becomes possible to make easily-bendable in this direction. As a result of this, the bendable tube 212A is bendable in a direction shifted from the Up side by the predetermined angle θ.

Five wires 400 to 404 are inserted into the bendable tube 212A. The wire 400 is positioned on the Up side corresponding to an upper direction of an image captured by the imager 214, the wire 401 is positioned on the Down side being an opposite side of the Up side, and the wire 402 and the wire 403 are positioned on the Right side orthogonal to the Up side, and the Left side being an opposite side of the Right side. If the wires 400 to 403 are pulled toward the proximal end side, the bendable tube 212A bends toward the Up side, the Down side, the Right side, or the Left side.

The wire 404 is positioned in a direction shifted from the Up side by the predetermined angle θ. The predetermined angle θ is 0°≤θ<90°, and is more preferably, 10°≤θ≤50°. If the wire 404 is pulled toward the proximal end side, by the bendable tube 212A bending in a direction shifted from the Up side by the predetermined angle θ, in the ultrasound endoscope 2 observing a digestive tract, it is possible to observe a biliopancreatic portion only by one wire operation using the ultrasound endoscope 2 inserted into duodenum. In addition, in the conventional ultrasound endoscope, it has been necessary to perform an operation of bending a bendable tube by operating a wire on the Up side, and rotating a distal end portion of an endoscope by a predetermined angle θ, and great care has been required. Nevertheless, a bendable tube of an endoscope other than an ultrasound endoscope may be made bendable in a direction shifted from the Up side by a predetermined angle θ. In this case, such an effect that, when the bendable tube is bent largely, an insertion tube does not appear at the center of an image or the like is caused.

In addition, in the above-described embodiment, the ultrasound endoscope 2 including the convex-type ultrasound transducer 7 has been described, but the ultrasound transducer may have a radial type or a linear type. Furthermore, the disclosure may be applied to an endoscope not including an ultrasound transducer.

According to the disclosure, it is possible to realize an endoscope that can realize a bending shape suitable for an intended purpose, while maintaining the operability.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscope comprising:
an operating portion configured to receive a bending operation performed by an operator;
an insertion tube that is provided on the operating portion and that has a central axis;
a bendable tube that is provided on a distal end side of the insertion tube and that is bendable in a curvature direction orthogonal to the central axis, the bendable tube including a plurality of bendable tube pieces that are arrayed along the central axis, adjacent bendable tube pieces being connected to each other by a pair of connection portions, the pair of connection portions being arranged line-symmetric with respect to the curvature direction, the bendable tube being configured to allow different positions in the curvature direction of the adjacent bendable tube pieces that are at least part of the plurality of bendable tube pieces;
a distal end rigid portion that is provided on a distal end side of the bendable tube; and
a wire having a first end connected to the operating portion, and a second end connected to either the bendable tube or the distal end rigid portion.

2. The endoscope according to claim 1, wherein positions in the curvature direction of the pair of connection portions are different between the distal end side of the bendable tube and a proximal end side of the bendable tube.

3. The endoscope according to claim 1, wherein positions in the curvature direction of the connection portions are positions getting away from the wire as getting close to the distal end side of the bendable tube.

4. The endoscope according to claim 1, wherein, in the bendable tube piece, a taper surface is formed in such a manner that a width of the bendable tube piece decreases along the curvature direction from the pair of connection portions.

5. The endoscope according to claim 1, wherein the second end of the wire is fixed to the bendable tube piece positioned closest to the distal end side of the bendable tube.

6. The endoscope according to claim 1,
wherein the pair of connection portions are a hinge, and
wherein the pair of connection portions are parallel to each other.

7. The endoscope according to claim 1, further comprising an imager configured to generate an image by imaging an observation object,
wherein the bendable tube is bendable toward an up side corresponding to an upper direction of the image, and is bendable toward a down side being an opposite side of the up side, and
wherein positions in the curvature direction of the pair of connection portions are positioned close to the down side as getting close to the distal end side of the bendable tube.

8. The endoscope according to claim 7, wherein the bendable tube is bendable toward a right side orthogonal to the up side, and bendable toward a left side being an opposite side of the right side.

9. The endoscope according to claim 8, wherein the pair of connection portions are arranged to face each other with centering around the central axis in a direction extending along the right side and the left side.

10. The endoscope according to claim 7, wherein the bendable tube is bendable in a direction shifted from the up side by a predetermined angle.

11. The endoscope according to claim 1, wherein positions in the curvature direction of the pair of connection portions are same in the pair of connection portions, and different for each of the bendable tube pieces.

12. The endoscope according to claim 1, further comprising an ultrasound transducer configured to transmit and receive ultrasound waves.

13. The endoscope according to claim 12, wherein the ultrasound transducer is a convex-type ultrasound transducer.
